(19) United States
(12) Patent Application Publication
(10) Pub. No.: US 2011/0021795 A1
(43) Pub. Date: Jan. 27, 2011

(54) PRODUCION METHOD OF PROPYLENE OXIDE

(75) Inventors: Hideo Kanazawa, Osaka (JP); Masahiko Mizuno, Nara (JP); Michio Yamamoto, Shiga (JP)

Correspondence Address: FOLEY AND LARDNER LLP;SUITE 500, 3000 K STREET NW, WASHINGTON, DC, 20007, US

(73) Assignee: SUMITOMO CHEMICAL COMPANY, LIMITED

(21) Appl. No.: 12/934,252

(22) PCT Filed: Mar. 26, 2009
(86) PCT No.: PCT/JP2009/056847

§ 371 Date: Sep. 23, 2010

(30) Foreign Application Priority Data
Mar. 28, 2008 (JP) 2008-086235
Dec. 22, 2008 (JP) 2008-325293

(51) Int. Cl. C07D 301/02

(52) U.S. Cl. 549/518

(57) ABSTRACT

A production method of propylene oxide, wherein hydrogen, oxygen and propylene are reacted by a multistep process in a mixed solvent of acetonitrile and water, in the presence of a layered precursor of Ti-MWW and a catalyst comprising palladium supported on a carrier.

### TECHNICAL FIELD

The present invention relates to a production method of propylene oxide.

### BACKGROUND ART

As a method for producing propylene oxide by reacting hydrogen, oxygen, and propylene gas in a liquid phase, there is known a method described in Patent Document 1.

### [Patent Document 1] JP No. 2002-511455 T

### DISCLOSURE OF THE INVENTION

However, the method described in Patent Document 1 was not necessarily a satisfactory method in terms of productivity of propylene oxide.

The present invention provides a production method of propylene oxide, wherein hydrogen, oxygen, and propylene are reacted by a multistep process in acetonitrile or a mixed solvent of acetonitrile and water in the presence of a layered precursor of Ti-MWW and a catalyst comprising palladium supported on a carrier.

According to the method of the present invention, productivity of propylene oxide can be improved.

### BEST MODES FOR CARRYING OUT THE INVENTION

The multi-step process in the present invention refers to a reaction process typically comprising n (n represents an integer of 2 or larger) reaction zones, where a part or whole of a reaction medium coming out of the (n−1)th reaction zone is fed to the nth reaction zone. Here, the reaction zones refer to zones where catalysts are contained and reactions are carried out and which are separated by zones where no reaction is conducted. A reactor may have one reaction zone or a plurality of reaction zones. For example, in the case of a slurry bed reactor, one reactor usually has one reaction zone. In the case of a fixed bed reactor, a plurality of reaction zones can be disposed in one reactor if the catalyst layer is separated by zones where no reaction is conducted. Reaction conditions for each reaction zone may be the same or different. The reaction medium refers to a liquid comprising, at least, propylene oxide and acetonitrile, and, further, in some cases, water. In addition to these, the medium may further comprise hydrogen, oxygen and propylene. The concentration of propylene oxide contained in the reaction medium fed to the nth reaction zone is usually higher than 0% but is 50% by weight or lower, preferably in the range of 0.1 to 20% by weight.

To the first reaction zone, usually, all of acetonitrile, water, propylene, hydrogen and oxygen are fed. To the nth reaction zone, at least one selected from acetonitrile, water, propylene, hydrogen and oxygen may be fed, in addition to a part or whole of the reaction medium coming out of the (n−1)th reaction zone.

According to the method of the present invention, the concentration of propylene oxide in the reaction medium coming out of the nth reaction zone can be usually made higher than the concentration of propylene oxide in the reaction medium fed to the nth reaction zone. When the concentration of the propylene oxide in the reaction medium fed to the nth reaction zone is, for example, higher than 0% by weight but 6.1% by weight or lower, the concentration of propylene oxide in the reaction medium coming out of the nth reaction zone can be made higher by at least 1.3% by weight than the concentration of propylene oxide in the reaction medium fed to the nth reaction zone. When the concentration of propylene oxide in the reaction medium fed to the nth reaction zone is, for example, higher than 6.1% by weight but lower than 10% by weight, the concentration of propylene oxide in the reaction medium coming out of the nth reaction zone can be made higher than the concentration of propylene oxide in the reaction medium fed to the nth reaction zone. Even when the propylene oxide concentration in the reaction medium fed to the nth reaction zone is 10% by weight or higher, the concentration of propylene oxide in the reaction medium coming out of the nth reaction zone can be made higher than the concentration of propylene oxide in the reaction medium fed to the nth reaction zone. In this way, by connecting n reaction zones, a reaction liquid containing a high concentration of propylene oxide can be usually obtained, or the propylene oxide production reaction proceeds to produce more propylene oxide in the reaction medium containing propylene oxide at appreciable concentration.

When the concentration of propylene oxide is high, the amount of acetonitrile to be recycled can be reduced, making it possible to cut down the energy required for recycling, to an economical advantage. Thus, the concentration of propylene oxide in the reaction medium coming out of the nth reaction zone is preferably 1% by weight or higher, more preferably 3% by weight or higher, even more preferably 6% by weight or higher. The upper limit of the concentration of propylene oxide is not particularly limited, but it is usually 60% by weight or lower, preferably 30% by weight or lower, depending on the activity of catalyst.

Propylene used in the reaction of the present invention includes one produced by, for example, thermal decomposition, heavy oil contact cracking, or catalytic reforming of methanol. The propylene may be either purified propylene or crude propylene which did not particularly go through a purification step. As the propylene, propylene having a purity of usually 90% by volume or higher, preferably 95% by volume or higher is used. Such propylene is exemplified by one which contains, in addition to propylene, for example, propane, cyclopropane, methylacetylene, propadiene, butadiene, butanes, butenes, ethylene, ethane, methane or hydrogen.

There are various embodiments for the forms of propylene supplied depending on the reaction pressure, but there is no particular limitation. Propylene may be supplied either in a gaseous form or in a liquid form. It is preferable that propylene is fed to the reaction, dissolved in an organic solvent or in a mixed solvent of organic solvent and water by mixing before entering the reactor. Or, it is also preferable that, separately from the solvent, propylene alone is fed to the reactor as a liquid. The propylene subjected to the reaction may contain gaseous components such as nitrogen gas and hydrogen gas. The feed ratio of propylene to each reaction zone is not particularly limited.

In the reaction of the present invention, acetonitrile or a mixed solvent of acetonitrile and water is used as the reaction medium. The weight ratio of water and acetonitrile is usually in the range of 0:100 to 50:50, preferably in the range of 10:90 to 40:60.

The amount of the mixture of acetonitrile and water fed per 1 part by weight of propylene is usually in the range of 0.02 to 70 parts by weight, preferably 0.2 to 20 parts by weight, more preferably 1 to 10 parts by weight. The feed ratio to each reaction zone is not particularly limited but it is preferable to feed 90% or more of the total propylene supply to the first reaction zone.

Acetonitrile may be either crude acetonitrile produced as a byproduct in the production process of acrylonitrile or purified acetonitrile. Usually purified acetonitrile having a purity of 95% or higher, preferably 99% or higher, more preferably 99.9% or higher is used. Typically, the crude acetonitrile contains, in addition to acetonitrile, for example, water, acetone, acrylonitrile, oxazole, allyl alcohol, propionitrile, hydrocyanic acid, ammonia, and a trace amount of copper and iron.

As molecular oxygen, oxygen purified by cryogenic separation, oxygen purified by PSA (a pressure swing adsorption method) or air may be used. The amount of oxygen fed is usually in the range of 0.005 to 10 moles, preferably 0.05 to 5 moles per 1 mole of propylene fed. The feed ratio of oxygen to each reaction zone is not particularly limited.

The method of preparation of hydrogen is not particularly limited but, for example, one produced by steam reforming of hydrocarbons is used. Usually, hydrogen of a purity of 80% by volume or higher, preferably 90% by volume or higher, is used. The amount of hydrogen fed is usually in the range of 0.05 to 10 moles, preferably 0.05 to 5 moles, per 1 mole of propylene fed. The feed ratio of hydrogen to each reaction zone is not particularly limited.

Usually, it is preferable, from a viewpoint of safety and disaster prevention, that the composition of supplied gas is kept outside the explosion ranges of hydrogen and propylene and that, for that purpose, diluent gas is accompanied in the reaction. Examples of the diluent gas include nitrogen, argon, methane, ethane, propane, carbon dioxide and the like. Preferable among these are nitrogen and propane, and more preferable is nitrogen. Regarding the amount of gas fed, when the explosion range is avoided by a hydrogen concentration, the concentration of hydrogen in the supplied gas is usually 3.9% by volume or lower; in that case, the concentration of oxygen can be any if it is equal to or lower than the critical oxygen concentration of propylene and is usually 11.5% by volume or lower, preferably 9% by volume or lower; to realize such a composition, the concentration is balanced by the diluent gas. When the explosion range is avoided by an oxygen concentration, the concentration of oxygen in the supplied gas is usually 4.9% by volume or lower, preferably 4% by volume or lower; in that case, there is no particular limitation on the hydrogen concentration or propylene concentration but usually the concentrations of both hydrogen and propylene are 10% by volume or lower; to realize such a composition, the concentrations are balanced by the diluent gas.

As the layered precursor of Ti-MWW, preferable is a layered precursor of Ti-MWW having an X-ray diffraction pattern with the following values and also having a composition represented by the formula: xTiO₂.(1−x)SiO₂ (in the formula, x represents a number from 0.0001 to 0.1).

X-ray diffraction pattern:
Lattice spacing d/Å (angstrom)

13.2±0.6

12.3±0.3

9.0±0.3

6.8±0.3

3.9±0.2

3.5±0.1

3.4±0.1

The layered precursor of Ti-MWW can be prepared by methods described in, for example, Chemistry Letters, 774-775 (2000); Chemical Communications, 1026-1027 (2002); or JP No. 2003-327425 A.

Carriers for the catalyst comprising palladium supported on a carrier usually include oxides such as silica, alumina, titania, zirconia and niobia; hydrates such as niobic acid, zirconic acid, tungstic acid and titanic acid; carbons such as activated carbon, carbon black, graphite and carbon nanotubes; or titanosilicates. Preferable among these are carbons or titanosilicates, and more preferable is activated carbon or a layered precursor of Ti-MWW.

Palladium may be supported on a carrier by impregnating the carrier after preparing a palladium colloid solution or by impregnating the carrier after palladium salt is dissolved in a solvent. The palladium salts include, for example, palladium chloride, palladium nitrate, palladium sulfate, palladium acetate and palladium tetraamine chloride. When supported on the carrier using the colloid solution, it is usually preferable to calcinate the carrier under an inert gas atmosphere after supporting. When supported on the carrier using palladium salts, the catalyst is usually used after reduction by a reducing agent in a liquid phase or in a vapor phase. When supported on the carrier using palladium tetraamine chloride, it is possible, after supporting, to reduce the catalyst by the ammonia evolved by thermal decomposition thereof in the presence of an inert gas.

The amount of palladium supported is, based on the catalyst having palladium supported on a carrier, usually in the range of 0.0001 to 20% by weight, preferably 0.001 to 5% by weight. The catalyst having palladium supported on a carrier may contain one or more kinds of noble metals other than palladium. The noble metals other than palladium include platinum, ruthenium, rhodium, iridium, osmium and gold. There is no restriction on the content of the noble metals other than palladium.

The modes of reaction include a batch system, a slurry-bed continuous flow system, and a fixed-bed continuous flow system. Among these, the slurry-bed continuous flow system and a fixed-bed continuous flow system are preferable from a standpoint of productivity. In the case of the slurry-bed continuous flow system, both the titanosilicate catalyst and the catalyst having palladium supported on a carrier are filtered on a filter installed inside or outside the reactor and remain in the reactor. A portion of the catalyst in the reactor is either continuously or intermittently withdrawn and regenerated and, thereafter, the reaction is carried out while returning the restored catalyst to the reactor. Or, the reaction may be carried out while withdrawing a portion of the catalyst out of the reactor and adding a new titanosilicate catalyst and a catalyst having palladium supported on a carrier to the reactor in amounts corresponding to the amounts withdrawn.

At least one of the titanosilicate catalysts or the catalysts having palladium supported on carriers is preferably charged to every reaction zone. The amount of the catalyst charged in the reactor is usually in the range of 0.01 to 20% by weight, preferably 0.1 to 10% by weight, based on the reaction medium.

In the case of the fixed-bed continuous flow system, usually the reaction is carried out with the reaction and catalyst regeneration repeated alternately. In that case, it is preferable to use a catalyst molded using a molding agent and the like.

The feed ratios of hydrogen and oxygen which are fed to each reaction zone are not particularly limited. The reaction temperature is usually in the range of 0 to 150° C., preferably 20 to 100° C., more preferably 40 to 70° C. The reaction temperature of each reaction zone may be the same or different.

The reaction pressure, in absolute pressure, is usually in the range of 0.1 to 201 MPa, preferably 1 to 10 MPa. The reaction pressure in each reaction zone may be the same or different. Usually, from a viewpoint of transfer of the reaction liquid gas, it is preferable that the pressure in the (n−1)th reaction zone is higher than that in the nth reaction zone.

The mixed solvent of acetonitrile and water, which is the reaction medium, may contain an ionic compound comprising a cationic portion and an anionic portion. The mixed solvent containing an ionic compound is preferable in that, therein, propylene oxide can be produced in higher selectivity. By adding an ionic compound to the mixed solvent of acetonitrile and water, generation of propane or propylene glycol as by-products can be suppressed. The cationic portion of the ionic compound includes, for example, an ammonium ion; alkali metal ions such as a sodium ion and potassium ion; alkaline earth metal ions such as a magnesium ion and calcium ion; and a hydrogen ion. In the ammonium ion, the hydrogen atom(s) of NH₄⁺ may be substituted by organic group(s) and it includes, in addition to NH₄⁺, an alkylammonium or alkylarylammonium ion. Examples of alkylammonium include tetramethylammonium, tetraethylammonium, tetra-n-propylammonium, tetra-n-butylammonium and cetyltrimethylammonium. Examples of alkylarylammonium include benzylammonium, dibenzylammonium, tribenzylammonium and phenethylammonium. The anionic portions of the ionic compounds include, for example, carboxylate ions such as a benzoate ion, formate ion, acetate ion, propionate ion, butyrate ion, valerate ion, caproate ion, caprylate ion or caprate ion; a phosphate ion, hydrogenphosphate ion, dihydrogenphosphate ion, hydrogenpyrophosphate ion, or pyrophosphate ion; a halide ion; a sulfate ion; a carbonate ion or hydrogencarbonate ion; or a hydroxide ion. Preferable cationic portions include an ammonium ion; alkali metal ions such as a sodium ion and potassium ion; and a hydrogen ion. Preferable anionic portions include carboxylate ions such as acetate and benzoate ions; a phosphate ion, hydrogenphosphate ion, dihydrogenphosphate ion; a hydrogencarbonate ion; and a sulfate ion.

Specific examples of the ionic compounds include ammonium sulfate, ammonium hydrogensulfate, ammonium carbonate, ammonium hydrogencarbonate, diammonium hydrogenphosphate, ammonium dihydrogenphosphate, ammonium phosphate, ammonium hydrogenpyrophosphate, ammonium pyrophosphate, ammonium chloride, ammonium nitrate, ammonium benzoate, ammonium acetate, benzoic acid, sodium benzoate, potassium benzoate, lithium benzoate, magnesium benzoate, calcium benzoate, acetic acid, sodium acetate, potassium acetate, lithium acetate, cesium acetate, rubidium acetate, magnesium acetate, calcium acetate, strontium acetate, barium acetate, phosphoric acid, sodium dihydrogenphosphate, potassium dihydrogenphosphate, lithium dihydrogenphosphate, calcium dihydrogenphosphate, disodium hydrogenphosphate, dipotassium hydrogenphosphate, magnesium hydrogenphosphate, calcium hydrogenphosphate, barium hydrogenphosphate, sodium phosphate, potassium phosphate, lithium phosphate, magnesium phosphate, calcium phosphate, barium phosphate, pyrophosphoric acid, sodium pyrophosphate, potassium pyrophosphate, magnesium pyrophosphate, calcium pyrophosphate, sodium hygrogenpyrophosphate, formic acid, sodium formate, potassium formate, lithium formate, cesium formate, rubidium formate, strontium formate, magnesium formate, calcium formate, barium formate, propionic acid, sodium propionate, potassium propionate, cesium propionate, calcium propionate, butyric acid, sodium butyrate, valeric acid, sodium valerate, capronic acid, sodium capronate, caprylic acid, sodium caprylate, carbonic acid, sodium carbonate, potassium carbonate, lithium carbonate, rubidium carbonate, cesium carbonate, magnesium carbonate, calcium carbonate, strontium carbonate, barium carbonate, sodium hydrogencarbonate, potassium hydrogencarbonate, cesium hydrogencarbonate, sodium hydroxide, potassium hydroxide, lithium hydroxide, cesium hydroxide, rubidium hydroxide, magnesium hydroxide, calcium hydroxide, strontium hydroxide, barium hydroxide, sodium sulfate, potassium sulfate, lithium sulfate, cesium sulfate, rubidium sulfate, magnesium sulfate, calcium sulfate, strontium sulfate, barium sulfate, hydrogen fluoride, sodium fluoride, potassium fluoride, lithium fluoride, cesium fluoride, rubidium fluoride, magnesium fluoride, calcium fluoride, barium fluoride, strontium fluoride, hydrogen chloride, sodium chloride, potassium chloride, lithium chloride, cesium chloride, rubidium chloride, magnesium chloride, calcium chloride, strontium chloride, barium chloride, hydrogen bromide, sodium bromide, potassium bromide, lithium bromide, cesium bromide, rubidium bromide, magnesium bromide, calcium bromide, barium bromide, strontium bromide, hydrogen iodide, sodium iodide, potassium iodide, lithium iodide, cesium iodide, rubidium iodide, magnesium iodide, calcium iodide, strontium iodide and barium iodide.

The pH of the mixed solvent of acetonitrile and water fluctuates upon addition of an ionic compound to the mixed solvent of acetonitrile and water, the reaction medium. Usually, the pH is in the range of 5 to 12, preferably 7 or higher, more preferably from 7 to 10. Here, the pH is calculated by measurement of electrode potential at 20° C. by immersing the electrodes to the acetonitrile/water mixed solvent which is used for the reaction, the electrodes used being a silver/silver chloride reference electrode with a 4 mol/L potassium chloride solution as the internal solution and a silver/silver chloride indicator electrode with an acetate buffer solution as the internal solution.

The amount of the ionic compound to be added is not particularly limited but the upper limit is the solubility thereof in the mixed solvent of acetonitrile and water.

In order to improve the amount of propylene oxide produced per unit time by the catalyst, in addition to selectivity thereof, it is more preferable to select, among the ionic compounds, one having an ammonium ion as the cationic portion. Specific examples of the ionic compound having an ammonium ion as the preferable cationic portion include the above exemplified ammonium sulfate, ammonium hydrogensulfate, ammonium carbonate, ammonium hydrogencarbonate, diammonium hydrogenphosphate, ammonium dihydrogenphosphate, ammonium phosphate, ammonium hydrogenpyrophosphate, ammonium pyrophosphate, ammonium chloride, ammonium nitrate, ammonium benzoate, or ammonium acetate; more preferable are ammonium sulfate, ammonium hydrogencarbonate, ammonium acetate, ammonium dihydrogenphosphate, diammonium hydrogenphosphate, ammonium phosphate and ammonium benzoate; even more preferable are ammonium dihydrogenphosphate, diammonium hydrogenphosphate, ammonium phosphate and ammonium benzoate.

When an ionic compound having an ammonium ion as the cationic portion is added to the mixed solvent of acetonitrile and water, it is preferable to adjust the pH to 7 or higher. By doing so, propylene oxide can be produced in higher yield and, also, in higher selectivity. The upper limit of the pH is usually 12.0 or lower, preferably 10.0 or lower. The pH is measured and calculated by the same method as described above.

The ammonium salts are usually fed to the reactor dissolved in a solvent. The lower limit of the amount fed is usually 1×10⁻⁷ mole or more, preferably 1×10⁻⁶ mole or more per 1 kg of the solvent. The upper limit depends on the solubility in the solvent but is usually 20 moles, preferably 2.0 moles.

Further, one quinoid compound or a mixture of plural quinoid compounds may be added to the mixed solvent of acetonitrile and water.

The quinoid compounds include two kinds, namely p-quinoid compounds and o-quinoid compounds. The quinoid compounds used in the present invention comprise both of these.

The quinoid compounds are exemplified by p-quinoid compounds represented by the following formula (1) and phenanthraquinone compounds:

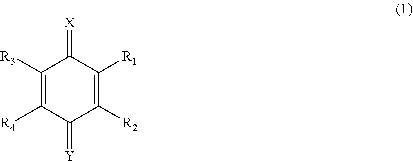

[In the formula (1), R₁, R₂, R₃, and R₄ represent a hydrogen atom; or neighboring R₁ and R₂ or R₃ and R₄ each independently are linked together at both ends and, together with the carbon atoms of the quinone skeleton to which they are bonded, form a benzene ring or a naphthalene ring, both of which may be substituted with an alkyl group or a hydroxyl group; X and Y may be the same or different from each other, and represent an oxygen atom or an NH group.]

The compounds represented by the formula (1) include:

1) a quinone compound (1A) represented by the formula (1), wherein R₁, R₂, R₃, and R₄ represent a hydrogen atom, and both X and Y represent an oxygen atom;
2) a quinoneimine compound (1B) represented by the formula (1), wherein R₁, R₂, R₃, and R₄ represent a hydrogen atom, X represents an oxygen atom, and Y represents an NH group;
3) a quinonediimine compound (1C) represented by the formula (1), wherein R₁, R₂, R₃, and R₄ represent a hydrogen atom, and X and Y represent an NH group.

The quinoid compounds represented by the formula (1) include anthraquinone compounds represented by the following formula (2):

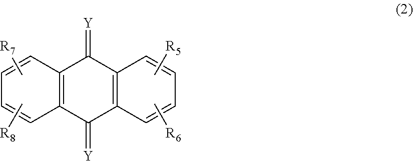

[In the formula (2), X and Y are as defined in the formula (1); R₅, R₆, R₇, and R₈ may be the same or different from each other and represent a hydrogen atom, a hydroxyl group, or an alkyl group (a C₁ to C₅ alkyl group such as, for example, methyl, ethyl, propyl, butyl and pentyl).]

In the formulae (1) and (2), X and Y preferably represent an oxygen atom. The quinoid compounds represented by the formula (1), wherein X and Y are an oxygen atom, are especially referred to as quinone compounds or p-quinone compounds. Also, the quinoid compounds represented by the formula (2), wherein X and Y are an oxygen atom, are further especially referred to as anthraquinone compounds.

Dihydro derivatives of the quinoid compounds include the compounds represented by the formulae (3) and (4), which are the dihydro derivatives of the compounds represented by the formulae (1) and (2):

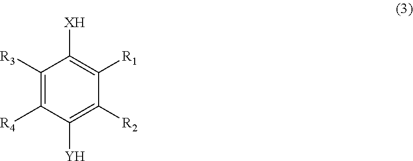

[In the formula (3), R₁, R₂, R₃, and R₄, X, and Y are as defined in relation to the formula (1)];

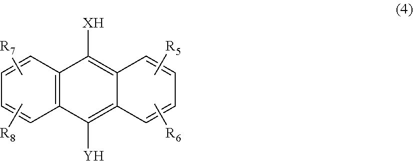

[In the formula (4), X, Y, R₅, R₆, R₇, and R₈ are as defined in relation to the formula (2)]
In the formulae (3) and (4), X and Y preferably represent an oxygen atom. The dihydro derivatives of the quinoid compound represented by the formula (3), wherein X and Y are an oxygen atom, are especially referred to as dihydroquinone compounds or p-dihydroquinone compounds. Also, the dihydro derivatives of the quinoid compounds represented by the formula (4), wherein X and Y are an oxygen atom, are further especially referred to as dihydroanthraquinone compounds.

Examples of phenanthraquinone compounds include 1,4-phenanthraquinone which is a p-quinoid compound, and 1,2-, 3,4-, and 9,10-phenathraquinones which are o-quinoid compounds.

Specific quinone compounds include benzoquinones, naphthoquinones, and anthraquinones, for example, 2-alkylanthraquinone compounds such as 2-ethylanthraquinone, 2-t-butyl anthraquinone, 2-amylanthraquinone, 2-methylanthraquinone, 2-butyl anthraquinone, 2-t-amylanthraquinone, 2-isopropyl anthraquinone, 2-s-butylanthraquinone or 2-s-amylanthraquinone; 2-hydroxyanthraquinone; polyalkylanthraquinone compounds such as 1,3-dimethylanthraquinone, 2,3-dimethylanthraquinone, 1,4-dimethylanthraquinone, or 2,7-dimethylanthraquinone; polyhydroxyanthraquinones such as 2,6-dihydroxyanthraquinone; naphthoquinone and its mixtures.

Preferable quinoid compounds include anthraquinone and 2-alkylanthraquinone compounds (in the formula (2), X and Y are an oxygen atom; R₅ is an alkyl group substituted at the 2 position; R₆ represents hydrogen, R₇ and R₈ represent a hydrogen atom). Preferable dihydro derivatives of quinoid compounds include the dihydro derivatives corresponding to these preferable quinoid compounds.

The method for adding the quinonoid compound or dihydro derivative of the quinoid compound (hereinafter, the latter is abbreviated as the quinoid compound derivative) to the reaction solvent includes a method whereby the quinoid compound derivative is dissolved in the liquid phase and, thereafter, used for the reaction. For example, the hydrogenated compound of the quinoid compound such as hydroquinone or 9,10-anthracenediol may be added to the liquid phase and may be used by generating a quinoid compound in the reactor by oxidation by oxygen.

Further, the quinoid compounds used in the present invention, including the quinoid compounds exemplified above, may be partially transformed into dihydro derivatives, which are hydrogenated quinoid compounds, depending on the reaction conditions. These compounds may also be used.

The quinoid compound is usually fed dissolved in acetonitrile to the reactor. The lower limit of the amount fed is usually 1×10⁻⁸ mole or more, preferably 1×10⁻⁷ mole or more, per 1 kg of the solvent. The upper limit depends on the solubility in the solvent, but is usually 10 moles, preferably 1.0 mole.

After the reaction, the reaction mixture is passed through a gas-liquid separation column, solvent separation column, crude propylene oxide separation column, propane separation column, and solvent purification column. Thus, the reaction mixture is separated into crude propylene oxide, a gaseous component mainly comprising hydrogen/oxygen/nitrogen, recovered propylene, recovered acetonitrile-water, and a recovered anthraquinone compound. The recovered propylene, recovered acetonitrile-water, and recovered anthraquinone are desirably fed to the reactor again and recycle-used for economic reasons. When such recycled propylene contains propane, cyclopropane, methylacetylene, propadiene, butadiene, butanes, butenes, ethylene, ethane, methane or hydrogen, it may be recycled after separation and purification, if necessary.

The recovered mixed solvent of acetonitrile and water may be used after separation and purification, if necessary, when it contains components represented by acetone, acrylonitrile, oxazole, allyl alcohol, propionitrile, propanol, 2,4-dimethyloxazoline or 2,5-dimethyloxazoline, which are byproducts produced in the reaction and have boiling points close to the azeotropic temperature of acetonitrile-water. The recovered anthraquinone may be used after separation and purification, if necessary, when it contains components represented by water, acetonitrile, anthracene compounds, anthrahydroquinone compounds, tetrahydroanthraquinone compounds, propylene glycol, acetamide, N-(2-hydroxypropane-1-yl)acetamide or N-(1-hydroxypropane-2-yl)acetamide, which are byproducts produced in the reaction and have boiling points higher than the azeotropic temperature of acetonitrile-water.

### EXAMPLES

Hereinafter, the present invention will be described in reference to Examples, but the present invention is not limited to these Examples.

### Reference Example 1

### Production Example of a Layered Precursor of Ti-MWW

The layered precursor of Ti-MWW used for the present reactions were produced as follows: In an autoclave, a gel was prepared by dissolving under stirring 112 g of TBOT (tetra-n-butylorthotitanate), 565 g of boric acid and 410 g of fumed silica (cab-o-sil M7D) in 899 g of piperidine and 2402 g of purified water at room temperature under an air atmosphere and, after aging for 1.5 hours, the autoclave was closed tightly. After raising the temperature over 8 hours under further stirring, a hydrothermal synthesis was carried out by maintaining the reaction mixture at 160° C. for 120 hours to obtain a suspended solution. The suspended solution obtained was filtered and the filter cake was washed with water until the pH of the filtrates became about 10. Then, the filter cake was dried at 50° C. to obtain white powder which still contained moisture. To 15 g of the powder obtained was added 750 ml of 2 N nitric acid, followed by reflux for 20 hours. Thereafter, the suspension was filtered and the filter cake was washed with water until the pH of the filtrates became nearly neutral, and was sufficiently dried at 50° C. to obtain 11 g of white powder. The X-ray diffraction pattern of this white powder was measured by the use of an X-ray diffraction apparatus using copper K-α radiation. As a result, it was confirmed that the white powder was a layered precursor of Ti-MWW, and the titanium content thereof according to an ICP emission analysis was 1.65% by weight.

### Reference Example 2

### Production Example of Ti-MWW

The layered precursor of Ti-MWW, obtained in Reference Example 1, was calcinated at 530° C. for 6 hours to obtain Ti-MWW catalyst powder. The fact that the powder obtained has an MWW structure was confirmed by measuring an X-ray diffraction pattern as in Reference Example 1. The titanium content according to the ICP emission analysis was 1.77% by weight.

### Reference Example 3

### Production Example of Palladium Supported Layered Precursor of Ti-MWW (1)

In an 1 L recovery flask, there was prepared a 300 mL aqueous solution containing 0.0902 mmol of palladium tetraamine chloride. To this aqueous solution was added 9 g of the layered precursor of Ti-MWW obtained in Reference Example 1 and the mixture was stirred for 8 hours. After completion of stirring, water was removed by a rotary evaporator and, further, the residue was vacuum dried at 80° C. for 4 hours. The catalyst precursor powder obtained was calcinated at 150° C. for 6 hours under a hydrogen atmosphere to obtain a palladium supported layered precursor of Ti-MWW. The palladium content according to an ICP emission analysis was 0.11% by weight.

### Reference Example 4

### Production Example of Palladium Supported Layered Precursor of Ti-MWW (2)

In an 1 L recovery flask, there was prepared a 300 mL aqueous solution containing 0.0847 mmol of a palladium colloid. To this aqueous solution was added 9 g of the layered precursor of Ti-MWW obtained in Reference Example 1 and the mixture was stirred for 8 hours. After completion of stirring, water was removed by a rotary evaporator and, further, the residue was vacuum dried at 80° C. for 8 hours. The catalyst precursor powder obtained was washed with 1 L of water and dried in vacuo again at 80° C. for 8 hours to obtain a palladium supported layered precursor of Ti-MWW. The palladium content according to an ICP emission analysis was 0.11% by weight.

### Example 1

As a reaction medium coming out of the (n−1)th reaction zone, acetonitrile-water containing 10% by weight of propylene oxide was prepared. This was fed to the nth reaction zone, the reaction was conducted, and an increase in the amount of propylene oxide at the outlet side of the nth reaction zone was investigated. In a 300 cc autoclave were charged 131 g of acetonitrile-water having a weight ratio of water/acetonitrile=30/70, 2.28 g of a layered precursor of Ti-MWW, and 0.198 g of an activated carbon-supported catalyst containing 1% palladium, the pressure was adjusted to 4 MPa in absolute pressure with nitrogen, and the temperature inside the autoclave was adjusted to 50° C. by circulating warm water through the jacket. To the autoclave were continuously fed mixed gas comprising 3.6% by volume of hydrogen, 2.1% by volume of oxygen, and 94.3% by volume of nitrogen at a rate of 146 NL/Hr; acetonitrile-water (with the weight ratio of water/acetonitrile being 30/70) comprising 0.7 mmol/kg of anthraquinone, 0.7 mmol/kg of ammonium dihydrogenphosphate, and 10.0% by weight of propylene oxide at a rate of 90 g/Hr; and liquid propylene containing 0.4% by volume of propane at a rate of 36 g/Hr. The pH of the mixed solvent of acetonitrile and water was 6.4. During the reaction, the reaction temperature was controlled at 50° C. and the reaction pressure at 4 MPa. The liquid component and gas component were continuously withdrawn, with the reaction mixture filtered through a sintered filter to remove solid components, the layered precursor of Ti-MWW and activated carbon-supported palladium catalyst, and after gas-liquid separation, the reaction mixture was returned to normal pressure. After 6 hours, samples of the reaction liquid and gas obtained were taken and the liquid and gas were each analyzed by gas chromatography. The concentration of propylene oxide in the resultant reaction liquid increased to 11.0% by weight. The increase in the amount of propylene oxide before and after the nth reaction zone including propylene oxide entrained by the reaction gas was 24 mmol/hr, increase in the amount of propylene glycol was L5 mmol/hr, and increase in the amount of propane was 6.3 mmol/hr.

### Example 2

The same operation as in Example 1 was carried out except that 1.98 g of Ti-MWW layered precursor-supported catalyst containing 0.1% by weight of palladium was used instead of the layered precursor of Ti-MWW and the activated carbon-supported catalyst containing 1% of palladium. The concentration of propylene oxide in the resultant reaction liquid increased to 10.7% by weight. The increase in the amount of propylene oxide before and after the reactor including propylene oxide entrained by the reaction gas was 19 mmol/hr.

### Comparative Example 1

The same operation as in Example 1 was carried out except that 2.28 g of Ti-MWW prepared in Reference Example 2 was used instead of the layered precursor of Ti-MWW. The concentration of propylene oxide in the resultant reaction liquid decreased to 9.2% by weight. The reason why the concentration of propylene oxide decreases is that the propylene oxide fed reacts with water in the reactor and is transformed into 1,2-propylene glycol. The increase in the amount of propylene oxide before and after the reactor including propylene oxide entrained by the reaction gas was 1.8 mmol/hr.

### Example 3

The same operation as in Example 1 was carried out except that acetonitrile-water containing 3.0% by weight of propylene oxide was prepared and used as the reaction medium. The concentration of propylene oxide in the resultant reaction liquid increased to 6.0% by weight. The increase in the amount of propylene oxide before and after the reactor including propylene oxide entrained by the reaction gas was 49 mmol/hr.

### Comparative Example 2

The same operation as in Comparative Example 1 was carried out except that acetonitrile-water containing 3.2% by weight of propylene oxide was prepared and used as the reaction medium. The concentration of propylene oxide in the resultant reaction liquid was 4.5% by weight. The increase in the amount of propylene oxide before and after the reactor including propylene oxide entrained by the reaction gas was 25 mmol/hr.

### Example 4

The same operation as in Example 1 was carried out except that acetonitrile-water containing 6.1% by weight of propylene oxide was prepared and used as the reaction medium. The concentration of propylene oxide in the resultant reaction liquid increased to 8.2% by weight. The increase in the amount of propylene oxide before and after the reactor including propylene oxide entrained by the reaction gas was 39 mmol/hr.

### Comparative Example 3

The same operation as in Comparative Example 1 was carried out except that acetonitrile-water containing 6.1% by weight of propylene oxide was prepared and used as the reaction medium. The concentration of propylene oxide in the resultant reaction liquid was 7.1% by weight. The increase in the amount of propylene oxide before and after the reactor including propylene oxide entrained by the reaction gas was 20 mmol/hr.

### Example 5

The same operation as in Example 1 was carried out except that acetonitrile-water (with the weight ratio of water/acetonitrile being 30/70) which did not contain ammonium dihydrogenphosphate but contained 0.7 mmol/kg of anthraquinone and 9.5% by weight of propylene oxide was used as the reaction medium. The pH of the acetonitrile-water mixed solvent fed to the reactor was 6.7. The concentration of propylene oxide in the resultant reaction liquid was found 9.4% by weight. The increase in the amount of propylene oxide before and after the nth reaction zone including propylene oxide entrained by the reaction gas was 16 mmol/hr, increase in the amount of propylene glycol was 4.8 mmol/hr, and increase in the amount of propane was 8.2 mmol/hr.

### Example 6

The same operation as in Example 5 was carried out except that an acetonitrile-water mixed solvent (with the weight ratio of water/acetonitrile being 30/70) containing 0.7 mmol/kg of anthraquinone, 3.0 mmol/kg of ammonium dihydrogenphosphate, and 10.4% by weight of propylene oxide was used as the reaction medium. The pH of the solvent fed to the reactor was 5.9. The concentration of propylene oxide in the resultant reaction liquid increased to 11.6% by weight. The increase in the amount of propylene oxide before and after the nth reaction zone including propylene oxide entrained by the reaction gas was 29 mmol/hr, increase in the amount of propylene glycol was 4.4 mmol/hr, and increase in the amount of propane was 6.5 mmol/hr.

### Example 7

The same operation as in Example 5 was carried out except that an acetonitrile-water mixed solvent (with the weight ratio of water/acetonitrile being 30/70) containing 0.7 mmol/kg of anthraquinone, 3.0 mmol/kg of diammonium hydrogenphosphate, and 10.0% by weight of propylene oxide was used as the reaction medium. The pH of the solvent fed to the reactor was 8.4. The concentration of propylene oxide in the resultant reaction liquid increased to 11.7% by weight. The increase in the amount of propylene oxide before and after the nth reaction zone including propylene oxide entrained by the reaction gas was 37 mmol/hr, increase in the amount of propylene glycol was 3.9 mmol/hr, and increase in the amount of propane was 5.8 mmol/hr.

### Example 8

The same operation as in Example 5 was carried out except that an acetonitrile-water mixed solvent (with the weight ratio of water/acetonitrile being 30/70) containing 0.7 mmol/kg of anthraquinone, 3.0 mmol/kg of ammonium phosphate, and 10.0% by weight of propylene oxide was used as the reaction medium. The pH of the solvent fed to the reactor was 8.6. The concentration of propylene oxide in the resultant reaction liquid increased to 11.7% by weight. The increase in the amount of propylene oxide before and after the nth reaction zone including propylene oxide entrained by the reaction gas was 35 mmol/hr, increase in the amount of propylene glycol was 2.5 mmol/hr, and increase in the amount of propane was 4.5 mmol/hr.

### Example 9

The same operation as in Example 5 was carried out except that an acetonitrile-water mixed solvent (with the weight ratio of water/acetonitrile being 30/70) containing 0.7 mmol/kg of anthraquinone, 3.0 mmol/kg of ammonium benzoate, and 10.1% by weight of propylene oxide was used as the reaction medium. The pH of the solvent fed to the reactor was 7.6. The concentration of propylene oxide in the resultant reaction liquid increased to 11.7% by weight. The increase in the amount of propylene oxide before and after the nth reaction zone including propylene oxide entrained by the reaction gas was 35 mmol/hr, increase in the amount of propylene glycol was 3.4 mmol/hr, and increase in the amount of propane was 5.5 mmol/hr.

### Example 10

The same operation as in Example 5 was carried out except that an acetonitrile-water mixed solvent (with the weight ratio of water/acetonitrile being 30/70) containing 0.7 mmol/kg of anthraquinone, 15.0 mmol/kg of ammonium benzoate, and 10.1% by weight of propylene oxide was used as the reaction medium. The pH of the solvent fed to the reactor was 7.7. The concentration of propylene oxide in the resultant reaction liquid increased to 11.7% by weight. The increase in the amount of propylene oxide before and after the nth reaction zone including propylene oxide entrained by the reaction gas was 35 mmol/hr, increase in the amount of propylene glycol was 2.9 mmol/hr, and increase in the amount of propane was 3.7 mmol/hr.

### Example 11

The same operation as in Example 5 was carried out except that an acetonitrile-water mixed solvent (with the weight ratio of water/acetonitrile being 30/70) containing 0.7 mmol/kg of anthraquinone, 3.0 mmol/kg of ammonium hydrogencarbonate, and 9.6% by weight of propylene oxide was used as the reaction medium. The pH of the solvent fed to the reactor was 8.8. The concentration of propylene oxide in the resultant reaction liquid increased to 10.2% by weight. The increase in the amount of propylene oxide before and after the nth reaction zone including propylene oxide entrained by the reaction gas was 28 mmol/hr, increase in the amount of propylene glycol was 3.8 mmol/hr, and increase in the amount of propane was 6.1 mmol/hr.

### Example 12

The same operation as in Example 5 was carried out except that an acetonitrile-water mixed solvent (with the weight ratio of water/acetonitrile being 30/70) containing 0.7 mmol/kg of anthraquinone, 3.0 mmol/kg of ammonium sulfate, and 9.7% by weight of propylene oxide was used as the reaction medium. The pH of the solvent fed to the reactor was 6.2. The concentration of propylene oxide in the resultant reaction liquid increased to 10.0% by weight. The increase in the amount of propylene oxide before and after the nth reaction zone including propylene oxide entrained by the reaction gas was 25 mmol/hr, increase in the amount of propylene glycol was 4.8 mmol/hr, and increase in the amount of propane was 6.9 mmol/hr.

### Example 13

The same operation as in Example 5 was carried out except that an acetonitrile-water mixed solvent (with the weight ratio of water/acetonitrile being 30/70) containing 0.7 mmol/kg of anthraquinone, 3.0 mmol/kg of ammonium acetate, and 9.5% by weight of propylene oxide was used as the reaction medium. The pH of the solvent fed to the reactor was 7.6. The concentration of propylene oxide in the resultant reaction liquid increased to 9.8% by weight. The increase in the amount of propylene oxide before and after the nth reaction zone including propylene oxide entrained by the reaction gas was 23 mmol/hr, increase in the amount of propylene glycol was 3.5 mmol/hr, and increase in the amount of propane was 4.5 mmol/hr.

### Example 14

The same operation as in Example 5 was carried out except that an acetonitrile-water mixed solvent (with the weight ratio of water/acetonitrile being 30/70) containing 0.7 mmol/kg of anthraquinone, 3.0 mmol/kg of dipotassium hydrogenphosphate, and 10.0% by weight of propylene oxide was used as the reaction medium. The pH of the solvent fed to the reactor was 9.6. The concentration of propylene oxide in the resultant reaction liquid increased to 10.9% by weight. The increase in the amount of propylene oxide before and after the nth reaction zone including propylene oxide entrained by the reaction gas was 18 mmol/hr, increase in the amount of propylene glycol was 3.3 mmol/hr, and increase in the amount of propane was 3.5 mmol/hr.

### Example 15

The same operation as in Example 5 was carried out except that an acetonitrile-water mixed solvent (with the weight ratio of water/acetonitrile being 30/70) containing 0.7 mmol/kg of anthraquinone, 3.0 mmol/kg of sodium benzoate, and 9.5% by weight of propylene oxide was used as the reaction medium. The pH of the solvent fed to the reactor was 8.5. The concentration of propylene oxide in the resultant reaction liquid increased to 9.6% by weight. The increase in the amount of propylene oxide before and after the nth reaction zone including propylene oxide entrained by the reaction gas was 17 mmol/hr, increase in the amount of propylene glycol was 4.3 mmol/hr, and increase in the amount of propane was 4.9 mmol/hr.

### Example 16

The same operation as in Example 5 was carried out except that an acetonitrile-water mixed solvent (with the weight ratio of water/acetonitrile being 30/70) containing 0.7 mmol/kg of anthraquinone, 3.0 mmol/kg of sodium acetate, and 9.5% by weight of propylene oxide was used as the reaction medium. The pH of the solvent fed to the reactor was 8.6. The concentration of propylene oxide in the resultant reaction liquid increased to 9.8% by weight. The increase in the amount of propylene oxide before and after the nth reaction zone including propylene oxide entrained by the reaction gas was 20 mmol/hr, increase in the amount of propylene glycol was 3.8 mmol/hr, and increase in the amount of propane was 4.6 mmol/hr.

### Example 17

The same operation as in Example 5 was carried out except that an acetonitrile-water mixed solvent (with the weight ratio of water/acetonitrile being 30/70) containing 0.7 mmol/kg of anthraquinone, 0.7 mmol/kg of disodium hydrogenphosphate, and 9.6% by weight of propylene oxide was used as the reaction medium. The pH of the solvent fed to the reactor was 9.3. The concentration of propylene oxide in the resultant reaction liquid increased to 9.9% by weight. The increase in the amount of propylene oxide before and after the nth reaction zone including propylene oxide entrained by the reaction gas was 21 mmol/hr, increase in the amount of propylene glycol was 4.1 mmol/hr, and increase in the amount of propane was 5.5 mmol/hr.

### Example 18

The same operation as in Example 1 was carried out except that acetonitrile-water mixed solvent (with the weight ratio of water/acetonitrile being 30/70) which did not contain anthraquinone but contained 0.7 mmol/kg of ammonium dihydrogenphosphate, and 11.0% by weight of propylene oxide was used as the reaction medium. The concentration of propylene oxide in the resultant reaction liquid increased to 11.5% by weight. The increase in the amount of propylene oxide before and after the reactor including propylene oxide entrained by the reaction gas was 16 mmol/hr.

### Example 19

The same operation as in Example 1 was carried out except that acetonitrile-water mixed solvent (with the weight ratio of water/acetonitrile being 30/70) which did not contain anthraquinone and an ionic compound but contained 11.0% by weight of propylene oxide was used as the reaction medium. The increase in the amount of propylene oxide before and after the reactor including propylene oxide entrained by the reaction gas was 4.0 mmol/hr.

### Comparative Example 4

The same operation as in Comparative Example 1 was carried out except that acetonitrile-water mixed solvent (with the weight ratio of water/acetonitrile being 30/70) which did not contain anthraquinone and an ionic compound but contained 11.0% by weight of propylene oxide was used as the reaction medium. The concentration of propylene oxide in the resultant reaction liquid decreased to 10.3% by weight. The amount of propylene oxide before and after the reactor including propylene oxide entrained by the reaction gas decreased to 1.6 mmol/hr.

### INDUSTRIAL APPLICABILITY

The present invention has a possibility of application in the production of propylene oxide.

1. A production method of propylene oxide, wherein hydrogen, oxygen and propylene are reacted by a multistep process in acetonitrile or a mixed solvent of acetonitrile and water, in the presence of a layered precursor of Ti-MWW and a catalyst comprising palladium supported on a carrier.

2. The production method according to claim 1, wherein the multistep process is a multistep process comprising n (n represents an integer of 2 or larger) reaction zones and a reaction medium comprising propylene oxide in a concentration of higher than 0% by weight but 6.1% by weight or lower is fed to the nth reaction zone.

3. The production method according to claim 1, wherein the multistep process is a multistep process comprising n (n represents an integer of 2 or larger) reaction zones and a concentration of propylene oxide in a reaction medium fed to the nth reaction zone is higher than 6.1% by weight but lower than 10% by weight.

4. The production method according to claim 1, wherein the multistep process is a multistep process comprising n (n represents an integer of 2 or larger) reaction zones and a concentration of propylene oxide in a reaction medium fed to the nth reaction zone is 10% by weight or higher.

5. The production method according to claim 1, wherein the layered precursor of Ti-MWW has an X-ray diffraction pattern with the following values and also has a composition represented by the general formula:
xTiO₂.(1−x)SiO₂
(in the formula, x represents a number from 0.0001 to 0.1);
X-ray diffraction pattern:
Lattice spacing d/Å (angstrom)
13.2±0.6
12.3±0.3
9.0±0.3
6.8±0.3
3.9±0.2
3.5±0.1
3.4±0.1.

6. The production method according to claim 1, wherein the carrier supporting palladium is activated carbon.

7. The production method according to claim 1, wherein the carrier supporting palladium is the layered precursor of Ti-MWW.

8. The production method according to claim 1, wherein the reaction medium is a mixed solvent of acetonitrile and water comprising an ionic compound which comprises a cationic portion and an anionic portion.

9. The production method according to claim 8, wherein the cationic portion of the ionic compound is an ammonium ion.

10. The production method according to claim 9, wherein pH of the mixed solvent of acetonitrile and water is 7 or higher, the mixed solvent comprising the ionic compound and being fed to the reaction zone.
